(19) United States
(12) Patent Application Publication
(10) Pub. No.: US 2016/0058542 A1
(43) Pub. Date: Mar. 3, 2016

(54) Chondroitin Sulfate Proteoglycan Containing Implants for Guided Nerve Regeneration and Methods of Manufacture Thereof

(71) Applicant: BACTERIN INTERNATIONAL, INC., Belgrade, MT (US)

(72) Inventors: Mark SCHALLENBERGER, Bozeman, MT (US); Helena LOVICK, Belgrade, MT (US)

(21) Appl. No.: 14/838,811

(22) Filed: Aug. 28, 2015

Related U.S. Application Data
(60) Provisional application No. 62/043,234, filed on Aug. 28, 2014.
(60) Provisional application No. 62/043,253, filed on Aug. 28, 2014.

(51) Int. Cl. A61F 2/02; A61L 27/28

(52) U.S. Cl. CPC A61F 2/02; A61L 27/28; A61L 2420/02; A61L 2430/32

(57) ABSTRACT

The invention relates to biocompatible implants with enhanced guided nerve axon regeneration and methods of manufacture thereof.

### CROSS-REFERENCE TO RELATED APPLICATIONS

This application claims the benefit of U.S. Provisional Patent Application No. 62/043,234, filed 28 Aug. 2014, and U.S. Provisional Patent Application No. 62/043,253, filed 28 Aug. 2014, both of which are hereby incorporated by reference in their entireties.

### FIELD OF THE INVENTION

The invention generally relates to chondroitin sulfate proteoglycan containing implants to promote guided nerve regeneration and methods of manufacture thereof. The invention relates to chondroitin sulfate proteoglycan (CSPG) containing implants manufactured by coating of implant materials with chondroitin sulfate proteoglycans, non-enzymatic treatment of biological nerve grafts to remove only a portion of the native chondroitin sulfate proteoglycans, or combinations of the foregoing. In some embodiments, the implants can be used for repair of peripheral nerve injuries.

### BACKGROUND

Over 550,000 peripheral nerve injuries occur each year in the United States affecting 5% of patients with a level 1 traumatic injury. These injuries are particularly devastating because they result in motor and sensory deficits with life altering outcomes. In addition to direct effects of the damage, the lost sensory and motor function places patients at significant risk for secondary injuries and represents one of the most common causes of lost productivity in adults.

Repair of nerve damage remains a significant challenge in reconstructive surgery. While human nerves possess the potential to regenerate following injury, the regenerative processes are dependent upon tissue continuity throughout the damaged nerve section. As such, injuries that result in nerve transections generally will not spontaneously regenerate and left untreated frequently lead to loss of motor and sensory function.

Current standard of care for reconstruction of transection injuries currently requires the use of interpositional nerve autografting, replacing the damaged nerve with a less critical nerve harvested from the patient. While autografting represents the gold standard treatment for complex peripheral nerve injuries, it presents a number of clinical limitations. Clinical limitations of autografting include donor site morbidity and limited supply.

Nerve allografts and xenografts, nerve grafts derived from deceased donor tissue, are promising substitutes for nerve autografts. By replacing the damaged nerve section with a nerve allograft or xenograft, the continuity of the nerve tissue is restored and nerve regeneration can proceed. Significant progress has been made in the field, but due to the substantial difficulty of processing nerve tissue, a product that yields functional recovery equivalent to autografts has yet to be commercialized.

U.S. Pat. No. 7,402,319 entitled “Cell-Free Tissue Replacement for Tissue Engineering,” which is incorporated by reference in its entirety, discloses methods of washing a native, acellular nerve tissue replacement in solutions including sulfobetaines and anionic surface-active detergents. The technology presented in U.S. Pat. No. 7,402,319 aims to enhance the regenerative capacity of the nerve grafts by removing the cellular material within the nerve graft rendering them non-immunogenic.

U.S. Pat. No. 7,732,200 entitled “Using Chondroitin Sulfate Proteoglycan (CSPG)-Degrading Enzymes Mixture to Propagate Nervous System Tissue in Culture for Use as Tissue Targeted Tools in Treatment of Brain and Neurodegenerative Disorders,” which is incorporated by reference in its entirety, discloses compositions and methods for culturing nerve tissue in vitro and nerve grafts produced thereby. The technology presented in U.S. Pat. No. 7,732,200 aims to enhance the regenerative capacity of nerve graft by eliminating chondroitin sulfate proteoglycans which have previously been shown to inhibit nerve axon growth. While this prior art discloses technology for enhancing the regenerative capacity of nerve grafts by removing at least a portion of the grafts chondroitin sulfate proteoglycans, the prior art relies upon enzymatic treatment that is costly and undesirable from a manufacturing and regulatory standpoint.

### SUMMARY OF THE INVENTION

The invention disclosed herein provides implants coated with chondroitin sulfate proteoglycan to enhance guided nerve axon regeneration and prevent nerve axon outgrowth. In some embodiments of the invention, the coating can be applied only to the exterior of the nerve graft and not the interior or the ends of the nerve graft, while in some embodiments, the nerve graft is fully coated. In some embodiments, the nerve graft has been decellularized and all of the native chondroitin sulfate proteoglycans have been removed prior to coating with chondroitin sulfate proteoglycans. Additionally, methods of manufacturing the described implants are disclosed.

The invention disclosed herein further provides a solution for costly and undesirable enzymatic treatment by treating biological nerve allografts in a non-enzymatic manner to remove at least a portion of the native chondroitin sulfate proteoglycans thus enhancing the regenerative capacity and performance of the biological nerve graft.

The invention disclosed herein provides biological nerve grafts in which at least a portion of their native chondroitin sulfate proteoglycans have been removed in a non-enzymatic manner. In some embodiments of the invention, the biological nerve graft can be a nerve allograft. In some embodiments of the invention, the biological nerve graft can also be processed to remove immunogenic material from the graft. In some embodiments, the non-enzymatic treatment removes only the side chains of the chondroitin sulfate proteoglycans leaving an intact, organized nerve sheath surrounding the nerve basal laminin.

It is one aspect of the present invention to provide an implant for repair of nerve damage, comprising a biocompatible scaffold, comprising an exterior surface, an interior surface, and two ends and defining a nerve growth path; and a coating comprising a chondroitin sulfate proteoglycan on at least a part of the exterior surface of the biocompatible scaffold.

In some embodiments, the coating is not on at least one of the interior surface and an end of the biocompatible scaffold.

In some embodiments, the biocompatible scaffold comprises at least one of a nerve autograft, a nerve allograft, and a nerve xenograft. In particular embodiments, the biocompatible scaffold is treated to remove substantially all chondroitin sulfate proteoglycans native to the biocompatible scaffold prior to being at least partially coated.

In some embodiments, the coating is positioned to inhibit growth of the nerve outside of the nerve growth path.

In some embodiments, the implant contains a nerve of biological origin.

In some embodiments, the implant comprises a material selected from the group consisting of metals, polymers, biological tissues, and combinations thereof.

It is another aspect of the present invention to provide a method for preparing a nerve implant, comprising coating a biocompatible scaffold, comprising an exterior surface, an interior surface, and two ends and defining a nerve growth path, with a composition comprising a chondroitin sulfate proteoglycan on at least a part of the exterior surface.

In some embodiments, the composition comprises at least one of a solution, a slurry, a suspension, an emulsion, a paste, a gel, and combinations thereof.

In some embodiments, the coating step is accomplished by at least one of spraying, dipping, rolling, and painting.

In some embodiments, the composition further comprises at least one of water, saline, a buffer, an organic solvent, and a polymer.

In some embodiments, the method further comprises drying the biocompatible scaffold by at least one of evaporation, heating, and lyophilization.

In some embodiments, the method further comprises freezing the biocompatible scaffold.

In some embodiments, the biocompatible scaffold is not coated on at least one end.

It is another aspect of the present invention to provide a method for preparing a nerve implant, comprising subjecting a nerve graft to a treatment process to remove at least a portion of chondroitin sulfate proteoglycans native to the nerve graft by non-enzymatic treatment, wherein the nerve graft comprises at least one of a nerve autograft, a nerve allograft, and a nerve xenograft.

In some embodiments, the treatment process comprises at least one of detergent treatment, temperature treatment, solvent treatment, selective hydrolysis, and mechanical treatment.

In some embodiments, the treatment process removes only side chains of the chondroitin sulfate proteoglycans native to the nerve graft, such that an organized nerve sheath surrounds at least one basal laminin of the nerve graft after the subjecting step.

It is another aspect of the present invention to provide a method for preparing a nerve implant, comprising providing a nerve graft, comprising an interior surface, an exterior surface, and two ends and defining a nerve growth path, the nerve graft being at least one of a nerve autograft, a nerve allograft, and a nerve xenograft; subjecting the nerve graft to a treatment process to remove at least a portion of chondroitin sulfate proteoglycans native to the nerve graft by non-enzymatic treatment; and coating the nerve graft with a composition comprising a chondroitin sulfate proteoglycan on at least a part of the exterior surface.

### DETAILED DESCRIPTION OF THE INVENTION

While the prior art discloses technology for enhancing the regenerative capacity of nerve grafts there remains a significant need to guide nerve axon regeneration within nerve grafts and prevent nerve axon outgrowth from the nerve grafts. It is currently understood in the literature that peripheral nerve repair via grafting is dependent on nerve axons growing from the proximal nerve stump across the nerve graft to make contact with the endonerual basal laminae in the distal nerve stump. As such, the degree in which sensory and motor function is restored is dependent not on the quantity of regenerating nerve axons within the nerve graft, but on the quantity of axons that successfully traverse the graft and enter into the distal nerve stump. An implant that guides the nerve axon regrowth would be desirable and result in increased functional recovery by increasing the percentage of nerve axons that successfully traverse the graft and make contact with the distal nerve stump. The implant would also be desirable by preventing nerve axon outgrowth, which has been associated with poor clinical outcomes in nerve grafting. Additionally, by guiding nerve axon regrowth the implant can be incorporated more rapidly. More rapid implant incorporation decreases the degradation time of the implant and surrounding tissues, further improving clinical outcome and functional recovery.

It has previously been shown that chondroitin sulfate proteoglycans present within nerve tissue are inhibitory to nerve axon regeneration and nerve allografts in which the chondroitin sulfate has been enzymatically digested induce increased nerve axon regrowth. However, the loss of this inhibitory cue often results in dysfunctional axon regrowth that fail to make contact with the distal nerve stump and often grow outward from the nerve graft.

The invention disclosed herein provides a solution for this axon regrowth challenge by coating the graft with chondroitin sulfate proteoglycans to prevent outgrowth and guide the regeneration of nerve axons.

The invention relates to implants for repair of nerve damage. An implant according to the present invention comprises a biocompatible scaffold, which can comprise an exterior surface, an interior surface, and two ends and define a nerve growth path. The biocompatible scaffold can be coated with a chondroitin sulfate proteoglycan on at least a part of the exterior surface to enhance nerve regeneration by guiding nerve axon regrowth and inhibiting nerve axon outgrowth. In some embodiments, the biocompatible scaffold can comprise biological tissue of animal origin, and can be a nerve autograft, a nerve allograft, or a nerve xenograft. In some embodiments, the nerve of biological origin can be decellularized and all, or substantially all (greater than about 95%), of the native chondroitin sulfate proteoglycans removed. In some embodiments of the invention, the coating of chondroitin sulfate proteoglycans can be applied only to the exterior of the biocompatible scaffold, or a portion thereof, and not the interior or the ends. In some embodiments, the biocompatible scaffold is a biologically-derived nerve graft non-enzymatically treated to remove at least a portion of the native chondroitin sulfate proteoglycans. In other embodiments, the biocompatible scaffold is non-enzymatically treated to remove at least a portion of the native chondroitin sulfate proteoglycans and is not further coated with chondroitin sulfate proteoglycan.

Those of ordinary skill in the art will understand that the term “chondroitin sulfate proteoglycan” refers not to a single compound but to a genus of compounds, each species of which comprises a core protein, generally a glycoprotein, and a glycosaminoglycan (GAG) sugar side chain attached by a covalent bond. The class of chondroitin sulfate proteoglycans includes, by way of non-limiting example, aggrecan, versican, neurocan, CSPG4, CSPG5, SMC3, brevican, CD44, and PTPRZ1. It is to be understood that the scope of the present invention encompasses coating a biocompatible scaffold with, and/or removing from a nerve graft, any one or more chondroitin sulfate proteoglycan compounds.

As used herein, “at least one,” “one or more,” and “and/or” are open-ended expressions that are both conjunctive and disjunctive in operation. For example, each of the expressions “at least one of A, B, and C,” “at least one of A, B, or C,” one or more of A, B, and C,” “one or more of A, B, or C,” and “A, B, and/or C” means A alone, B alone, C alone, A and B together, A and C together, B and C together, or A, B, and C together.

It is to be noted that the term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

“Allogenic” or “allograft,” as used herein, refers to tissue derived from a non-identical donor of the same species.

“Autogenic” or “autograft,” as used herein, refers to tissue derived from and implanted into the same identical patient.

“Chondroitin sulfate proteoglycan,” as used herein, is defined as a class of biological molecules consisting of a protein core and chondroitin sulfate side chains.

“Donor,” as used herein, refers to a living or deceased animal whose body is the source of biological tissue.

“End user,” as used herein, refers to a health care practitioner who performs the implantation of the implant into a patient.

“Nerve graft,” as used herein, refers to an implant within a patient to promote nerve regeneration following a nerve injury. In some embodiments, the nerve graft can be derived from the nerves of a donor.

“Patient,” as used herein, refers to a living recipient of the implant of the invention.

“Xenogenic” or “xenograft,” as used herein, is defined as tissue derived from a non-identical donor of a different species.

The biocompatible scaffold has an exterior surface, an interior surface, and two ends to define a nerve growth path that runs from a transected end of a nerve to a first end of the biocompatible scaffold, along the exterior and interior surfaces, to the second end of the biocompatible scaffold to another transected nerve end.

In some embodiments of the invention, the implants can be composed of biocompatible materials at least in part, such as biodegradable polymers, biocompatible plastics, biocompatible elastomers, biocompatible metals, and combinations thereof. In some embodiments, the implants can be composed of materials of biological origin at least in part, such as dermis, nerve tissue, or other collagen-based tissues. In some embodiments, the implants can be composed of nerves of biological origin at least in part. The nerves of biological origin can be autogenic, xenogenic, or allogenic. In some embodiments, the implant can be an allogenic nerve graft.

In further embodiments of the invention, the implants can consist of a biocompatible and or biodegradable conduit coated with chondroitin sulfate proteoglycans. In some embodiments, the conduit can serve to surround the ends of the transected nerves of the patient leaving a void between the transected nerves to further guide axon regrowth. In other embodiments, the conduit can surround the ends of the transected nerves of the patient can also contain material of biological origin in the center of the conduit. In some embodiments, the coating can be positioned to inhibit nerve growth outside of the nerve growth path. In preferred embodiments, the biocompatible conduit can contain allogenic nerve grafts. In some embodiments, the allogenic nerve grafts contained in the biocompatible conduit can be morselized.

Suitable biodegradable polymers include polycaprolactones, polyethylene glycols, polyhydroxyalkanoates, polyesteramides, polyglycolides, polyorthoesters, polyoxazolines, polyurethanes, polylactide-co-glycolides and combinations, and copolymers thereof. Suitable biocompatible plastics, elastomers, and metals include medical grade PVC, polyethylene, PEEK, polycarbone, polypropylene, polysulfone, polyurethane, silicone, Grade V titanium, 316LV stainless steel, combinations thereof and the like.

It is desirable for the biological tissues of the invention to be decellularized to reduce immunological response in the recipient of the implant. Methods for biological tissue decellularization include the use of detergents, solvents, freeze-thaw cycling, sonication, and many other techniques and combinations of the foregoing. For a review of several tissue decellularization techniques refer to Patnaik, et al. “Tissue Regeneration: Where Nano-Structure Meets Biology”, World Scientific Publishing Company, Singapore, 1^{st} Edition, Chapter 3, p. 8-12 (2014) and references cited therein, each of which is incorporated by reference in their entirety.

Additional benefits of decellularization include the removal of extraneous proteins and debris. As it is known that chondroitin sulfate proteoglycan are inhibitory molecules, which regulate axonal growth, in a preferred embodiment of the invention, all native chondroitin sulfate proteoglycans can be removed from biological tissue implants prior to coating of the implants with chondroitin sulfate proteoglycans. This removal of the native chondroitin sulfate proteoglycans serves to eliminate undesirable inhibition of axonal regrowth. The removal of chondroitin sulfate proteoglycans can be performed by methods such as chemical treatment, detergent treatment, sonication, combinations thereof and the like. In preferred embodiments, the removal of chondroitin sulfate proteoglycans can be achieved by non-enzymatic means.

Non-enzymatic removal of the chondroitin sulfate proteoglycans from biological tissues can be performed via selective hydrolysis of the sulfated glycosaminoglycan linkages. Selective hydrolysis can be performed via acidic, basic, hydrogen-bonding, ionic bonding, or other non-enzymatic catalytic means.

In other embodiments, the non-enzymatic removal of the chondroitin sulfate proteoglycans can be performed by soaking the biological tissues in organic solvents. The soak time can be optimized for the specific biological tissue type and the desired residual concentration of chondroitin sulfate proteoglycans to allow minimal disturbance of the extracellular matrix of the underlying tissue. Suitable solvent treatments include, but are not limited to, acetone, acetonitrile, chloroform, dichloromethane, dimethyl sulfoxide, ethanol, ethyl acetate, methanol, isopropanol, tetrahydrofuran, water, and mixtures thereof.

In other embodiments, the non-enzymatic removal of the chondroitin sulfate proteoglycans can be performed by subjecting the biological tissue to mechanical forces to break the sulfated glycosaminoglycan linkages. The mechanical forces include, but are not limited to, shaking, stirring, sonicating, or other agitations. The biological tissue can be subjected to these mechanical forces in the solid state (neat), in aqueous solution, in an alcohol-based solution, in an organic solution, or combinations of solution types. Additionally, the biological tissue can be subjected to these physical forces in the presence of a physical object that aids in breaking the sulfated glycosaminoglycan linkages. Suitable physical objects include but are not limited to inert glass beads, cationic ion exchange resins, anionic ion exchange resins, polar non-charged resins, or otherwise functional resins. In other embodiments the ion exchange resin can produce a local acidic, basic or otherwise ionic environment that effectively hydrolyzes the sulfated glycosaminoglycan linkages without damaging the remaining biological molecules and underlying extracellular matrix of the biological graft.

In other embodiments, the non-enzymatic removal of the chondroitin sulfate proteoglycans can be performed by exposing the biological tissue to a detergent.

In other embodiments, the non-enzymatic removal of the chondroitin sulfate proteoglycans can be performed by heating and/or cooling the biological tissue. By way of non-limiting example, the non-enzymatic removal of the chondroitin sulfate proteoglycans can be performed by freeze-thaw cycling, rapid cooling followed by rapid heating, and rapid heating followed by rapid cooling.

In some embodiments, the non-enzymatic treatment process may remove only the side chains of the native chondroitin sulfate proteoglycans. Removing only the side chains of the native chondroitin sulfate proteoglycans can be desirable, by way of non-limiting example, to ensure that an organized nerve sheath remains and surrounds the basal laminin or lamina of the biological tissue.

Subsequent to the removal of the native chondroitin sulfate proteoglycans (when applicable), the implants can be coated with chondroitin sulfate proteoglycan, for example, by application of a solution of chondroitin sulfate proteoglycan. The coating of the implants can be selective. In some embodiments, the coating of chondroitin sulfate proteoglycans can be exclusively applied to some or all of the exterior of the implants. In some embodiments, the exterior coating of chondroitin sulfate proteoglycans can be removed from the ends of the implant following the implant coating step. In preferred embodiments, the exterior coating of chondroitin sulfate proteoglycans may not be applied to the ends of the implant during the implant coating step. In either of these latter embodiments, the ends of the implant are not coated which allows for nerve regeneration between transected ends of a nerve along a nerve growth path from a transected nerve end to a first end of an implant through the biocompatible scaffold to a second end of the implant to another transected nerve end.

Methods of coating the biocompatible scaffold with chondroitin sulfate proteoglycans include first preparing a composition of chondroitin sulfate proteoglycans comprising at least one of a solution, a suspension, a slurry, an emulsion, a paste, and a gel. Following preparation of the composition of chondroitin sulfate proteoglycans, the biocompatible scaffold can be sprayed with the composition, dipped in the composition, rolled in the composition, painted with the composition, or coated with the composition by other related physical means. Alternatively, chondroitin sulfate proteoglycans can be applied as a paste or powder to the exterior surface of the biocompatible scaffold.

The liquid component of the compositions of chondroitin sulfate proteoglycans can include water, aqueous solutions, buffers, balanced salt solutions, saline, organic solvents, and polymers. Suitable water-based components include, but are not limited to, phosphate buffered saline, saline, or Hank's balanced salt solution. Alternatively, compositions of chondroitin sulfate proteoglycans can be formed in non-water based media such as water miscible or immiscible solvents including, but not limited to, acetone, acetonitrile, chloroform, dichloromethane, dimethyl sulfoxide, ethanol, ethyl acetate, methanol, isopropanol, tetrahydrofuran, and mixtures thereof. Furthermore, compositions of chondroitin sulfate proteoglycans can be composed of biphasic mixtures of water or water-based solutions and water miscible or immiscible solvents of the preceding water-based and non-water based listed media.

The coating compositions of chondroitin sulfate proteoglycans can also contain biodegradable polymers. Following the coating of the biocompatible scaffold with chondroitin sulfate proteoglycans, the resultant implant can be dried by evaporation, heating, lyophilization, or combinations thereof. In some embodiments, following coating of the biocompatible scaffold, the implant can be dried and then stored frozen. In some embodiments, following coating of the biocompatible scaffold, the implant can be left undried and then stored frozen. In some embodiments, following coating of the implant, the implant can be immediately used for nerve repair by an end-user.

Suitable biodegradable polymers for the coating include polycaprolactones, polyethylene glycols, polyhydroxyalkanoates, polyesteramides, polyglycolides, polyorthoesters, polyoxazolines, polyurethanes, poly(lactide-co-glycolide)s and combinations, and copolymers thereof. When the coating contains biodegradable polymers, the contained chondroitin sulfate proteoglycans within the coating can be released as the polymer degrades. In some embodiments, the release of the chondroitin sulfate proteoglycans from the coating can be performed at a desired rate for optimal nerve regeneration based on the judicious selection of the biodegradable polymer type and concentration.

While the embodiments and examples of the invention described herein have been characterized as being preferred, those of ordinary skill in the art will understand and appreciate the existence of variations, combinations, and equivalents of the specific embodiments, methods, and examples. The invention should therefore not be limited by the above described embodiments, methods, and examples, but by all embodiments and methods within the scope and spirit of the invention as claimed.

1. An implant for repair of nerve damage, comprising:
a biocompatible scaffold, comprising an exterior surface, an interior surface, and two ends and defining a nerve growth path; and
a coating comprising a chondroitin sulfate proteoglycan on at least a part of the exterior surface of the biocompatible scaffold.

2. The implant of claim 1, wherein the coating is not on at least one of the interior surface and an end of the biocompatible scaffold.

3. The implant of claim 1, wherein the biocompatible scaffold comprises at least one of a nerve autograft, a nerve allograft, and a nerve xenograft.

4. The implant of claim 3, wherein the biocompatible scaffold is treated to remove substantially all chondroitin sulfate proteoglycans native to the biocompatible scaffold and then is at least partially coated.

5. The implant of claim 1, wherein the coating is positioned to inhibit growth of nerve outside of the nerve growth path.

6. The implant of claim 1, wherein the implant comprises a material of biological origin.

7. The implant of claim 1, wherein the implant comprises a material selected from the group consisting of metals, polymers, biological tissues, and combinations thereof.

8. A method for preparing a nerve implant, comprising:
coating a biocompatible scaffold, comprising an exterior surface, an interior surface, and two ends and defining a nerve growth path, with a composition comprising a chondroitin sulfate proteoglycan on at least a part of the exterior surface.

9. The method of claim 8, wherein the composition comprises at least one of a solution, a slurry, a suspension, an emulsion, a paste, a gel, and combinations thereof.

10. The method of claim 8, wherein the coating step is accomplished by at least one of spraying, dipping, rolling, and painting.

11. The method of claim 8, wherein the composition further comprises at least one of water, saline, a buffer, an organic solvent, and a polymer.

12. The method of claim 8, further comprising drying the biocompatible scaffold by at least one of evaporation, heating, and lyophilization.

13. The method of claim 8, further comprising freezing the biocompatible scaffold.

14. The method of claim 8, wherein the biocompatible scaffold is not coated on at least one end.

15. A method for preparing a nerve implant, comprising:
subjecting a nerve graft to a treatment process to remove at least a portion of chondroitin sulfate proteoglycans native to the nerve graft by non-enzymatic treatment,
wherein the nerve graft comprises at least one of a nerve autograft, a nerve allograft, and a nerve xenograft.

16. The method of claim 15, wherein the treatment process comprises at least one of detergent treatment, temperature treatment, solvent treatment, selective hydrolysis, and mechanical treatment.

17. The method of claim 15, wherein the treatment process removes only side chains of the chondroitin sulfate proteoglycans native to the nerve graft, such that an organized nerve sheath surrounds at least one basal laminin of the nerve graft after the subjecting step.

18. A method for preparing a nerve implant, comprising:
providing a nerve graft, comprising an interior surface, an exterior surface, and two ends and defining a nerve growth path, the nerve graft being at least one of a nerve autograft, a nerve allograft, and a nerve xenograft;
subjecting the nerve graft to a treatment process to remove at least a portion of chondroitin sulfate proteoglycans native to the nerve graft by non-enzymatic treatment; and
coating the nerve graft with a composition comprising a chondroitin sulfate proteoglycan on at least a part of the exterior surface.
